(19) United States
(12) Patent Application Publication
(10) Pub. No.: US 2025/0065075 A1
(43) Pub. Date: Feb. 27, 2025

(54) OVERMOLD TECHNIQUE FOR PEEL-AWAY INTRODUCER DESIGN

(71) Applicants: ABIOMED, Inc., Danvers, MA (US); Oscor, Inc., Palm Harbor, FL (US)

(72) Inventors: Glen Fantuzzi, Danvers, MA (US); Michael Tafone, Danvers, MA (US); Jeffrey Drum, Palm Harbor, FL (US)

(73) Assignees: ABIOMED, Inc., Danvers, MA (US); Oscor, Inc., Palm Harbor, FL (US)

(21) Appl. No.: 18/788,325

(22) Filed: Jul. 30, 2024

Related U.S. Application Data
(63) Continuation of application No. 18/211,610, filed on Jun. 20, 2023, now Pat. No. 12,076,497.
Application No. 18/211,610 is a continuation of application No. 17/741,627, filed on May 11, 2022, now Pat. No. 11,717,640.
Application No. 17/741,627 is a continuation of application No. 15/834,783, filed on Dec. 7, 2017, now Pat. No. 11,364,363.
(60) Provisional application No. 62/431,671, filed on Dec. 8, 2016.

(51) Int. Cl. A61M 25/00; A61M 25/06; B29C 45/14; B29L 31/00

(52) U.S. Cl. CPC A61M 25/0009; A61M 25/0014; A61M 25/0668; B29C 45/14065; B29C 45/14336; A61M 25/0097; B29L 2031/7542

(57) ABSTRACT

A method for manufacturing a medical introducer includes placing an introducer sheath onto a mandrel, and overmolding an introducer hub onto a proximal end of the introducer sheath. The introducer sheath has one or more score lines formed on an inner surface and the mandrel has a number of surface protrusions so that when the introducer sheath is positioned on the mandrel, each of the surface protrusions contacts one of the score lines formed on the introducer sheath. The surface protrusions on the mandrel prevent plastic material from the introducer hub from contacting the score lines thereby maintaining the score lines during the overmolding.

### CROSS-REFERENCE TO RELATED APPLICATIONS

The present application is a continuation of U.S. patent application Ser. No. 18/211,610, filed Jun. 20, 2023, now allowed, which is a continuation of U.S. patent application Ser. No. 17/741,627, filed May 11, 2022, now U.S. Pat. No. 11,717,640, which is a continuation of U.S. patent application Ser. No. 15/834,783, filed Dec. 7, 2017, now U.S. Pat. No. 11,364,363, which claims the benefit of the filing date of U.S. Provisional Patent Application No. 62/431,671, filed on Dec. 8, 2016, the entire disclosures of which are hereby incorporated herein by reference.

### BACKGROUND OF THE INVENTION

Peel-away introducers are disposable medical devices used in a cardiac catheterization or other medical setting to deliver medical devices into the vasculature. Standard peel-away introducers include a proximal plastic hub coupled to a sheath. Medical devices can be inserted through the plastic hub and into the sheath, through which the device can be placed in a patient's body. Intravascular medical devices, such as intracardiac blood pumps, catheters, guidewires, or leads, can be introduced into a patient's vasculature through a peel-away introducer. Once the medical device has been positioned, the peel-away introducer can be removed. In one approach, the operator breaks the introducer by cracking the plastic introducer hub and peeling down the shaft of the sheath body. In order to break the peel-away introducer, the operator grasps the molded hub and breaks it at the proximal end of the sheath along axial notches or scorings. The sheath tears along perforations or scorings down one or both sides of the sheath and can be peeled axially. The peel-away introducer allows the introducer to be removed after a medical device is inserted into a patient through the introducer without disturbing or removing the medical device.

In a common manufacturing technique, peel-away introducers are formed from an extruded plastic tube with axial notches in a hub body. A hub body including notches and/or wings to facilitate breaking is overmolded onto an extruded plastic sheath or tube. The plastic tube is first assembled over a corepin or mandrel which defines and maintains the inner cavity of the hub and inner diameter and geometry of the sheath. The mandrel and extruded sheath are then placed into a mold cavity allowing for molten plastic to be injected and cooled, creating the plastic hub that the user breaks to initiate peeling.

In the overmolding technique, after the mandrel and extruded sheath are placed into a mold, molten plastic in injected into the mold. The plastic cools around the sheath in the shape of the mold, forming the hub body. The injected plastic is maintained at a high temperature when it is introduced to the mold, and the heat from the injected plastic surrounding the sheath may be sufficient to heat a top layer of the sheath plastic such that the plastic of the sheath may start to flow in a process called reflowing. The portion of the sheath disposed inside the hub of the assembly may lose its internal scoring or notching during the molding process if the heat of the injected plastic reflows into one or more of the scores/notches. As a result, the forces required to break the hub may be excessive or inconsistent because the sheath does not include a weakened section of one of the scores/notches. Furthermore, when the plastic of the sheath is reflowed and the scoring is removed in the section of the sheath within the hub, there is an increased risk that during the breaking of the hub the tear will not propagate to the scoring in the sheath body outside the section in the overmolded hub leading to a defective peel-away introducer sheath.

### SUMMARY OF THE INVENTION

Described herein are methods and systems for producing a peel-away introducer including an overmolded hub while maintaining the scoring of the sheath. During overmolding, a mandrel designed with outer surface protrusions on which the sheath is assembled, maintains the inner scorings of the sheath through the injection molding process despite the application of extreme heat. With the internal scorings of the sheath maintained, when the hub is broken by an operator, the proximal section of the sheath tube in the overmolded hub breaks more easily and with less required force. Sheaths manufactured by the methods disclosed herein can also peel more easily.

In one aspect, a method for manufacturing a medical introducer includes placing an introducer sheath onto a mandrel, and overmolding an introducer hub onto a proximal end of the introducer sheath. The introducer sheath has one or more score lines formed on an inner surface. The mandrel has a number of surface protrusions so that when the introducer sheath is positioned on the mandrel, each of the surface protrusions contacts one of the score lines formed on the introducer sheath and prevents plastic material from the introducer hub from contacting the score lines.

In some implementations, the number of surface protrusions on the mandrel is equal to the number of score lines formed on the sheath. In some implementations, the introducer hub includes a number of notches. In some implementations, the number of notches in the introducer hub is equal to the number of score lines formed on the sheath and also equal to the number of surface protrusions on the mandrel. In some implementations, at least one of the number of notches is aligned with a score line formed on the sheath. In some implementations, at least two notches are aligned with a score line formed on the sheath. In some implementations, the introducer hub and introducer sheath are configured to be broken into at least two pieces along the notches and score lines.

In some implementations, the number of surface protrusions on the mandrel is not equal to the number of score lines formed on the sheath. In some implementations, at least one of the number of score lines formed on the inner surface of the introducer sheath is removed during the overmolding. In some implementations, the number of surface protrusions on the mandrel is equal to a desired number of score lines in the sheath.

Variations and modifications will occur to those of skill in the art after reviewing this disclosure. The disclosed features may be implemented, in any combination and subcombination (including multiple dependent combinations and subcombinations), with one or more other features described herein. The various features described or illustrated above, including any components thereof may be combined or integrated in other systems. Moreover, certain features may be omitted or not implemented.

### BRIEF DESCRIPTION OF THE DRAWINGS

The foregoing and other objects and advantages will be apparent upon consideration of the following detailed description, taken in conjunction with the accompanying drawings, in which like reference characters refer to like parts throughout, and in which:

FIG. 1A shows a top view of an introducer hub molded over a sheath during the manufacture process according to an embodiment;

FIG. 1B shows a perspective view of a mandrel having both continuous surface protrusions and surface protrusions configured as a series of protrusions;

FIG. 2 shows a perspective view of a mandrel being inserted into a sheath during the manufacture process according to an embodiment;

FIG. 3 shows a perspective view of a mandrel fully inserted into a sheath during the manufacture process according to an embodiment;

FIG. 4 shows a perspective view of a mandrel having a surface protrusion according to an embodiment;

FIG. 5 shows a perspective view of a peel-away introducer hub assembly according to an embodiment;

FIG. 6 shows an alternative view of a peel-away introducer hub according to an embodiment; and

FIG. 7 shows a method for manufacturing a peel-away introducer hub according to an embodiment.

### DETAILED DESCRIPTION OF THE DRAWINGS

Following below are more detailed descriptions of various concepts related to inventive methods of manufacturing a peel-away medical introducer. It should be appreciated that various concepts introduced above and discussed in greater detail below may be implemented in any number of ways, as the disclosed concepts are not limited to any particular manner of implementation. For example, though peel-away introducer sheaths are depicted herein having two axial notches which divide the introducer hub and underlying sheath into two sections, the introducer hub and sheath may be manufactured to have any number of axial notches or perforations so as to allow parting into any number of portions along an axial direction. Furthermore, although surface protrusions of a mandrel are described with reference to maintaining score lines on a sheath for use in a peel-away introducer, the surface protrusions on the mandrel may be used to protect other features of the internal geometry of a sheath during any overmolding or heat-treating process. Examples of implementations and applications are provided primarily for illustrative purposes.

The methods and systems described herein enable production of a peel-away introducer including an overmolded hub while maintaining the scoring of the sheath. The mandrel for holding the hub and the sheath can have one or more outer elongate protrusions formed along an outer surface thereof. A protrusion can contact each of the corresponding inner scorings of the sheath so that when heat is applied the score lines are maintained through the injection molding process rather than filled with material from the hub or the sheath (e.g., plastic that has melted during heat application). With the internal scorings of the sheath maintained, when the hub is broken by an operator, the proximal section of the sheath tube in the overmolded hub breaks more easily and with less required force than if this material had flowed into the score lines and hardened. Additionally, with the score lines in the introducer sheath maintained, the hub can be broken and the sheath peeled longitudinally to facilitate removal of the introducer hub assembly. The overmolding method described herein promotes appropriate breaking of the introducer hub and peeling of the sheath as designed.

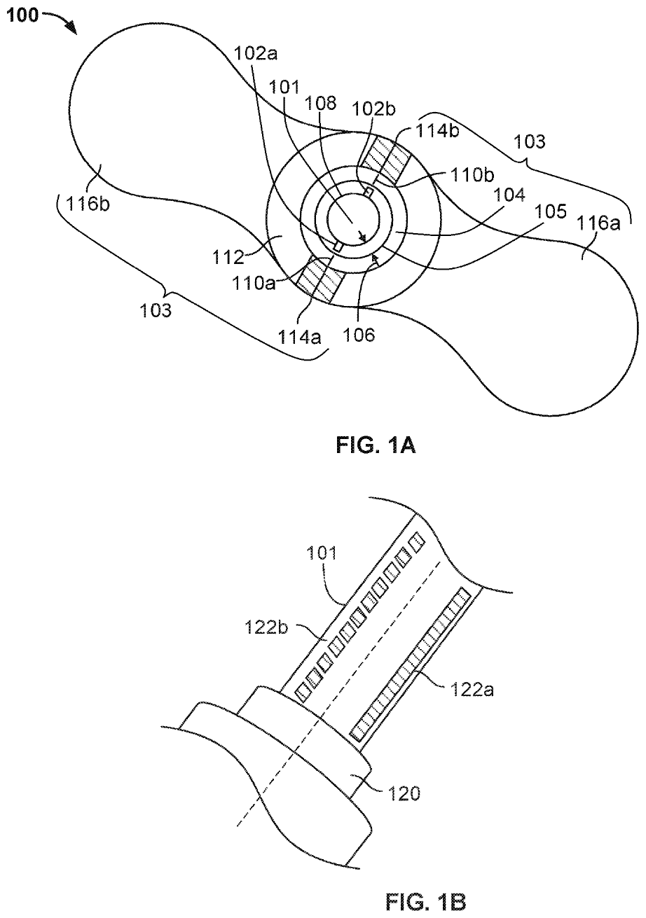

FIG. 1A shows a top view of an introducer hub and mandrel assembly 100 including a hub body 112 molded over an introducer sheath 104 during the manufacture process. The introducer hub assembly 103 includes an introducer sheath 104, a first score line 110a, a second score line 110b, a hub body 112, a first notch 114a, a second notch 114b, a first wing 116a, and a second wing 116b. The first score line 110a and the second score line 110b are on opposite sides of the introducer sheath 104. The introducer sheath 104 is positioned over a mandrel 101 (also called a corepin) having a first surface protrusion 102a and a second surface protrusion 102b on opposite sides of the mandrel 101. The first surface protrusion 102a and the second surface protrusion 102b are configured to coincide with the first score line 110a and the second score line 110b in the introducer sheath 104. After the introducer sheath 104 is positioned over the mandrel 101 with the first surface protrusion 102a and the second surface protrusion 102b aligned with the first score line 110a and the second score line 110b in the introducer sheath 104, the introducer hub body 112 can be overmolded over the proximal end of the introducer sheath 104. The first notch 114a and the second notch 114b are aligned with the first score line 110a and the second score line 110b in the introducer sheath 104 to facilitate breaking of the introducer hub assembly 103.

The mandrel 101 with the first surface protrusion 102a and the second surface protrusion 102b is shaped to maintain the inner geometry of the introducer sheath 104. The introducer sheath 104 fits tightly over the mandrel 101 in preparation for overmolding of the introducer hub assembly 103. There may be some distance 106 between an outer surface 108 of the mandrel 101 and an inner surface 105 of the introducer sheath 104, but in most cases the distance 106 is minimized such that the outer surface 108 of the mandrel 101 is in contact with the inner surface 105 of the introducer sheath 104 in areas which are to be protected during overmolding, such as at the first score line 110a and the second score line 110b. In some implementations, the distance 106 between the outer surface 108 of the mandrel 101 and the inner surface 105 of the introducer sheath 104 in areas which are not protected during overmolding is e.g., 0 mm, 0.1 mm, 0.2 mm, 0.3 mm, or 0.4 mm or it can be structured at any other suitable distance. The surface protrusions 102a and 102b may be set at a desired height from the body of the mandrel 101. In some implementations, the surface protrusions 102a and 102b extend from the surface of the mandrel 101 by about 0.05 mm, 0.1 mm, 0.2 mm, 0.3 mm, or any suitable height. In certain embodiments, the distance between the outer surface 108 of the mandrel 101 and an inner surface 105 of the introducer sheath 104 is between about 0 mm and 0.4 mm, while the protrusions extend from the outer surface 108 of the mandrel 101 and have a height of about 0.05 mm to 0.3 mm. In some implementations, the surface protrusions have a shape which is square, rectangular, cylindrical, or any other suitable shape. In some implementations, the protrusions extend longitudinally along the mandrel. In some implementations, the protrusions extend in a helix along the mandrel.

With the introducer sheath 104 fit over the mandrel 101, the introducer hub assembly 103 can be formed onto the sheath 104 such as by being injection molded over the introducer sheath 104. The injection molding process exposes the proximal end of the introducer sheath 104 to high temperatures and/or pressures which can cause reflow of the plastic from which the introducer sheath 104 is formed. Reflowing plastic may cover or completely fill the first score line 110a and the second score line 110b in the introducer sheath 104 if the first score line 110a and the second score line 110b are not protected by a corresponding protrusion. The first surface protrusion 102a and the second surface protrusion 102b extend into the first score line 110a and the second score line 110b such that even if the introducer sheath 104 is heated and reflow occurs, the first score line 110a and the second score line 110b are maintained.

If the first score line 110a and the second score line 110b are altered or removed by reflow of the plastic during construction, the introducer sheath 104 may not separate properly during breaking of the introducer hub assembly 103 after use. The hub body 112 may break as designed at first notch 114a and second notch 114b when an outward or downward force is applied to the first wing 116a and second wing 116b. However, if the first score line 110a and the second score line 110b have been damaged, the separation may not propagate to the introducer sheath 104, or from a proximal portion of the introducer sheath 104 to a more distal portion not in contact with the introducer hub assembly 103. As a result, it may be difficult or uncomfortable for an operator to apply the additional or excessive force required in order to initiate the breaking of the introducer hub assembly 103.

Though the introducer sheath 104 is depicted here having two score lines, the first score line 110a and the second score line 110b, the introducer sheath 104 may be configured with any number of score lines. In some implementations, the introducer sheath is configured with one score line, two score lines, three score lines, four score lines, six score lines, ten score lines, or any other suitable number of score lines. The score lines may be formed as perforations, scorings, indentations or by any other suitable means.

The mandrel 101 is depicted here having two surface protrusions, the first surface protrusion 102a and the second surface protrusion 102b, but may have any suitable number of surface protrusions. In some implementations, the mandrel 101 has one surface protrusion, two surface protrusions, three surface protrusions, four surface protrusions, six surface protrusions, or any other suitable number of surface protrusions. The mandrel 101 may have a number of surface protrusions equal to the number of score lines on the introducer sheath 104. Alternatively, the mandrel 101 may have a number of surface protrusions which is less than the number of score lines on the introducer sheath 104. If the number of surface protrusions is less than the number of score lines on the introducer sheath 104, any additional score lines on the introducer sheath 104 may be removed by reflow of material during the manufacture of the introducer hub assembly 103 overlay. For example, if an introducer sheath 104 has three score lines, but only two are desired for an introducer hub assembly a mandrel with only two surface protrusions may be used to protect two of the three score lines on the introducer sheath during overmolding. Alternatively, the mandrel 101 may have a number of surface protrusions that is greater than the number of score lines on the introducer sheath 104 such that only some of the total surface protrusions contact the score lines. This can allow, for example, the mandrel 101 to be configured to assemble sheaths having varying number of score lines.

The surface protrusions on the mandrel 101 may be matched to the type of score lines on the introducer sheath 104, as shown in FIG. 1B. FIG. 1B includes a mandrel 101 extending from a base 120 with a first surface protrusion 122a and a second surface protrusion 122b. The first surface protrusion 122a is configured as a continuous protrusion from the outer surface of the mandrel 101 which would contact a score line of an introducer sheath along an entire length without interruption. The second surface protrusion 122b is configured as a series of surface protrusions arranged in a line along the mandrel 101. The second surface protrusion 122b contacts the introducer sheath only at each of the individual protrusions, and not at the space between the protrusions. For example, a series of surface protrusions such as second surface protrusion 122b may be used to protect a score line configured as a perforation.

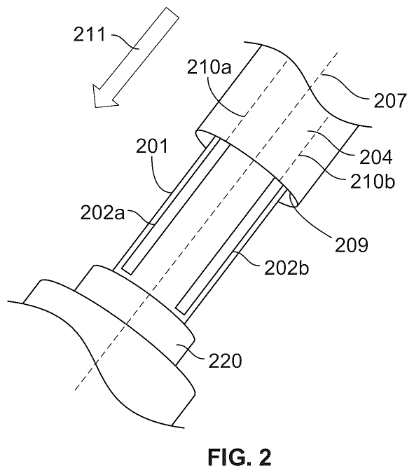

FIG. 2 shows a perspective view of a mandrel 201 partially inserted into an introducer sheath 204 in preparation for overmolding of an introducer hub. The mandrel 201 includes a base 220, a first surface protrusion 202a and a second surface protrusion 202b. The first surface protrusion 202a and a second surface protrusion 202b are parallel to a longitudinal axis 207 of the mandrel 201. The introducer sheath 204 includes a proximal end 209, a first score line 210a and a second score line 210b aligned parallel to the longitudinal axis 207 of the mandrel 201 which coincides with a longitudinal axis 207 of the introducer sheath 204 which the introducer sheath 204 is placed on the mandrel 201. The first surface protrusion 202a and a second surface protrusion 202b are aligned with the first score line 210a and the second score line 210b of the introducer sheath 204 in order to protect the score lines and internal geometry of the introducer sheath 204 during overmolding of an introducer hub at the proximal end 209 of the introducer sheath 204. Introducer sheath 204 is inserted over the mandrel 201 in the direction of arrow 211 prior to the overmolding of the introducer hub.

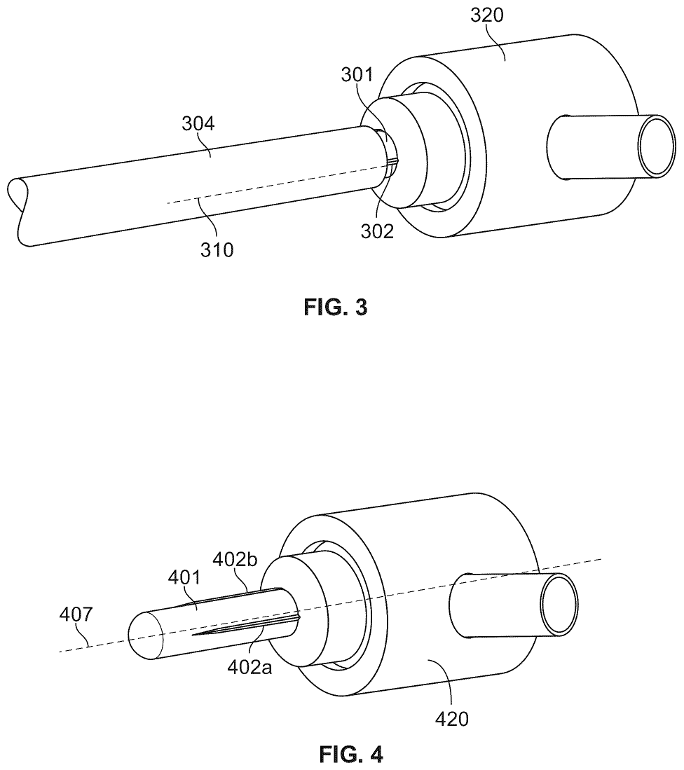

FIG. 3 shows a perspective view of a mandrel 301 fully inserted into an introducer sheath 304 in a final configuration prior to overmolding of an introducer hub onto the introducer sheath 304. The mandrel 301 includes a base 320 and one or more surface protrusions 302. The introducer sheath 301 includes a first score line 310 aligned parallel to a longitudinal axis 307 of the introducer sheath 304. The surface protrusion 302 is aligned with the first score line 310 to protect the first score line 310 from reflow during further processing. The introducer sheath 304 is designed to tear along the first score line 310 when the introducer hub assembly is broken. However, the first score line 310 may become damaged or be removed by the heat from the overmolding of the introducer hub body if the first score line 310 and any other score lines are not protected and maintained. The surface protrusion 302 of the mandrel 301 protects the internal geometry of the introducer sheath 304 including the first score line 310 during the overmolding of the introducer hub body so that the first score line 310 remains. The mandrel 301 is sized to fit inside the introducer sheath 304 and the surface protrusion 302 on the mandrel is likewise sized to align with and correspond to the first score line 310 on the introducer sheath 304.

FIG. 4 shows a perspective view of a mandrel 401 having surface protrusions 402a and 402b. The mandrel 401 includes a base 420, a first surface protrusion 402a, and a second surface protrusion 402b. The first surface protrusion 402a and the second surface protrusion 402b extend along the cylindrical mandrel 401 parallel to the longitudinal axis. The first surface protrusion 402a and the second surface protrusion 402b are sized and positioned to correspond to the internal geometry and score lines of the introducer sheath so that the internal geometry and space of the introducer sheath is maintained during the manufacture of the introducer hub on the introducer sheath.

Although two surface protrusions 402, the first surface protrusion 402a and the second surface protrusion 402b, are shown in FIG. 4, the mandrel 401 can have any suitable number or orientation of surface protrusions to protect and maintain score lines on an introducer sheath placed over the mandrel 401. The mandrel 401 may have one, two, three, four, six, eight, or any suitable number of surface protrusions. In some implementations, the mandrel 401 includes the same number of surface protrusions as the number of score lines on the introducer sheath. In some implementations, the mandrel 401 includes a smaller number of surface protrusions than the number of score lines on the introducer sheath. In such cases, the additional score lines on the introducer sheath will be removed by reflow of the plastic of the sheath during the overmolding process. Removal of unnecessary score lines during overmolding by not protecting the score lines with the mandrel may allow multiple styles of introducer hub assemblies to be prepared using a single type of introducer sheath. The surface protrusions may be aligned along the longitudinal axis 407 as shown here, or they may be oriented at an angle to the longitudinal axis or in a spiral orientation around the outer surface of the mandrel.

The first surface protrusion 402a and the second surface protrusion 402b shown in FIG. 4 are depicted as continuous raised sections of the mandrel 401. In some implementations, the surface protrusions on the mandrel 401 are configured as a series of raised dots in order to protect the specific geometry of the interior of the introducer sheath. In some implementations, the surface protrusions may be a continuous raised section configured as a ridge with an angled top, a squared top or a rounded top. The shape of the surface protrusions may be matched to a shape of the score lines in the introducer sheath.

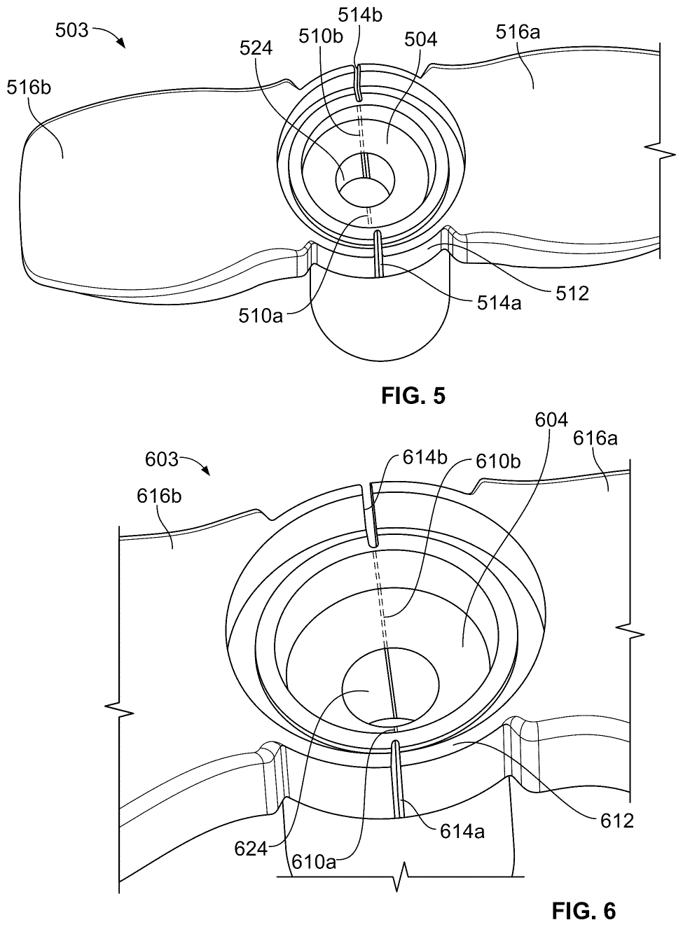

FIG. 5 shows a perspective view of a peel-away introducer hub assembly 503. The peel-away introducer hub assembly 503 includes a hub body 512, a first wing 516a, a second wing 516b, a first notch 514a, a second notch 514b, an introducer sheath 504, a first score line 510a, a second score line 510b, and a throughgoing cavity 524. The first notch 514a is aligned with the first score line 510a, and the second notch 514b is aligned with the second score line 510b. The first wing 516a and the second wing 516b extend on opposite sides of the hub body 512, such that the first notch 514a and the second notch 514b are positioned between the first wing 516a and the second wing 516b.

A medical device or catheter may be inserted into a patient through the throughgoing cavity 524 in the introducer hub assembly 503. After the device or catheter has been inserted, an operator can break the introducer hub assembly 503 by applying a force to the first wing 516a and the second wing 516b, breaking the introducer hub assembly 503 into two or more pieces. The introducer hub assembly 503 is designed to break at the first notch 514a and the second notch 514b upon the application of this force on the first wing 516a and the second wing 516b. The alignment of the first notch 514a and the second notch 514b with the first score line 510a and the second score line 510b, respectively, allows the break force applied by the operator to break the hub body 512 to propagate from the first notch 514a to the first score line 510a and from the second notch 514b to the second score line 510b to allow the introducer hub assembly 503 to be broken into two pieces and the introducer sheath 504 to be peeled away. Good alignment of the notches with the score lines that have been protected from damage during the overmolding of the hub body 512 allows the introducer hub assembly to be easily broken and peeled with a nominal breaking force applied.

FIG. 6 shows an alternative view of the peel-away introducer hub 503 of FIG. 5, illustrating the alignment of the second notch 614b with the second score line 610b opposite the first notch 614a and first score line 610a. The first wing 616a, second wing 616b, and cavity 624 of FIG. 6 are the same features described above with respect to first wing 516a, second wing 516b, and cavity 524 of FIG. 5, respectively. The alignment of the second notch 614b with the second score line 610b (and first notch 614a and first score line 610a) allows the transfer of the breaking force from the hub body 612 to the introducer sheath 604. If the score line 610 of the introducer sheath 604 has not been damaged or removed by the flow of plastic material from the introducer sheath 604 during the overmolding of the introducer hub body 612, the force may be propagated from the hub body 612 to the introducer sheath 604 without incident.

If the score line 610 of the introducer sheath 604 has been damaged or removed by the reflow of plastic material from the introducer sheath 604 during overmolding, the introducer sheath 604 may not easily split apart at the hub body 612 or below the hub body 612. This may require additional or excessive force to be applied to the hub body 612 or to the introducer sheath 604 in order to break the hub body 612 and introducer sheath. Alternatively, the malfunction of the introducer hub assembly 603 due to damaged score lines in the introducer sheath 604 may result in a defective introducer hub assembly which cannot be peeled away as designed. Alternative or suboptimal methods for removal may then be used which may require additional tools.

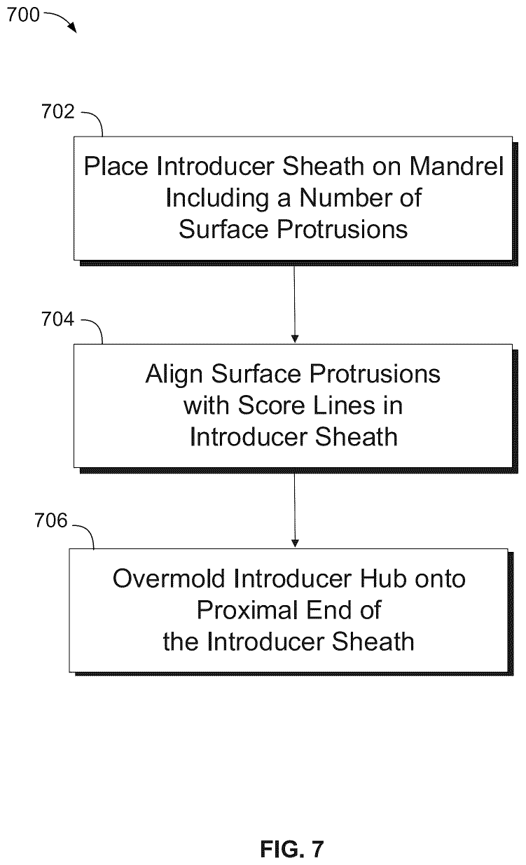

FIG. 7 shows a method 700 for manufacturing a peel-away introducer hub, such as the introducer hub assembly 103 of FIG. 1A, hub assembly 503 of FIG. 5, or any other suitable hub assembly. At step 702 the method includes placing an introducer sheath on a mandrel including a number of surface protrusions. The surface protrusions are designed to contact score lines in an interior surface of the introducer sheath in order to protect the geometry of the interior of the introducer sheath including the score lines. The mandrel may include the same number of surface protrusions as the number of score lines on the introducer sheath, or in some implementations, the mandrel includes a smaller number of surface protrusions if a number of score lines are designed to be removed during the next step. The mandrel and extruded sheath are then placed into a mold cavity allowing for molten plastic to be injected and cooled.

At step 704, the surface protrusions of the mandrel are aligned with the score lines of the introducer sheath. The introducer sheath fits tightly over the mandrel and the score lines on the interior of the introducer sheath are in contact with the surface protrusions of the mandrel. In some implementations, the surface protrusions of the mandrel may be aligned with the score lines of the introducer sheath before the mandrel is inserted into the introducer sheath, or simultaneous to insertion. The shape, size, and height of the surface protrusions may be matched to the score lines in the introducer sheath. In some implementations, the surface protrusions may be a continuous raised section configured as a ridge with an angled top, a squared top or a rounded top. The surface protrusions on the mandrel may be a variety of heights from the body of the mandrel to match the score lines in the introducer sheath. In some implementations, the surface protrusions extend from the surface of the mandrel by 0.05 mm, 0.1 mm, 0.2 mm, 0.3 mm, or any suitable height. In some implementations, there is some distance between an outer surface of the mandrel and an inner surface of the introducer sheath, but in most cases the distance is minimized such that the outer surface of the mandrel is in contact with the inner surface of the introducer sheath at least in regions which are to be protected from reflow during further manufacture. In some implementations, the distance between the outer surface of the mandrel and the inner surface of the introducer sheath in areas which are not to be protected is 0 mm, 0.1 mm, 0.2 mm, 0.3 mm, 0.4 mm or any other suitable distance. In some implementations, the surface protrusions 102a and 102b extend from the surface of the mandrel 101 by about 0.05 mm, 0.1 mm, 0.2 mm, 0.3 mm, or any suitable height. In certain implementations, the distance between the outer surface of the mandrel and an inner surface of the introducer sheath is between about 0 mm and 0.4 mm, while the protrusions extend from the outer surface of the mandrel and have a height of about 0.05 mm to 0.3 mm.

At step 706, the method includes overmolding an introducer hub onto a proximal end of the introducer sheath. The overmolding process uses heating, which may cause the plastic material of the introducer sheath to melt and reflow. The areas of the score lines which are protected by their contact with the surface protrusions of the mandrel are not covered by reflowing plastic and are maintained throughout the overmolding process. Areas which are not protected by the surface protrusions on the mandrel may reflow and fill in any additional score lines.

The overmolding of the introducer hub may also include overmolding one or more wings onto the introducer hub to aid in the breaking of the hub after use. After the introducer hub is overmolded onto the proximal end of the sheath the introducer hub and introducer sheath are removed from the mandrel. The introducer sheath includes a number of score lines which have been maintained by the mandrel during the overmolding of the introducer hub and introducer hub body. The score lines correspond to notches in the overmolded introducer hub. The introducer hub may be separated into two or more pieces at the notches and score lines after use with the application of a nominal force on the wings. Once the introducer hub has been separated at the notches and score lines, the introducer sheath may be peeled away along the score lines to remove the introducer hub assembly.

The foregoing is merely illustrative of the principles of the disclosure, and the methods and systems can be practiced other than the described implementations, which are represented for purposes of illustration and not of limitation. It is to be understood that the methods and systems disclosed herein, while shown for use in manufacture of a peel-away introducer hub and sheath, may be applied to other systems in which internal geometries of a sheath must be maintained during heat treatment.

Variations and modifications will occur to those of skill in the art after reviewing this disclosure. For example, the geometries and orientations of the surface protrusions of the mandrel can be beneficially chosen to match an internal geometry of the introducer sheath, or to complement an internal geometry or desired internal geometry. The disclosed features may be implemented, in any combination and subcombination (including multiple dependent combinations and subcombinations), with one or more other features described herein. The various features described or illustrated above, including any components thereof, may be combined or integrated in other systems. Moreover, certain features may be omitted or not implemented.

Examples of changes, substitution, and alterations are ascertainable by one skilled in the art and could be made without departing from the scope of the information disclosed herein.

1-23. (canceled)

24. A method of manufacturing a medical introducer, comprising:
placing an introducer sheath on a mandrel comprising a plurality of surface protrusions, the introducer sheath having a plurality of score lines formed on an inner surface thereof; and
overmolding a plastic material onto a proximal end of the introducer sheath to form an introducer hub using a mold surrounding the proximal end of the introducer sheath,
heating the plastic material wherein the plastic material is heated to such a temperature that the plastic material reflows, and
reflowing the plastic material,
wherein, when the introducer sheath is positioned on the mandrel, a number of the plurality of surface protrusions in contact with the plurality of score lines is less than a total number of score lines on the introducer sheath and the score lines of the plurality of score lines that are not in contact with the surface protrusions are removed by the reflowing of plastic material.

25. The method of claim 24, wherein the number of the plurality of surface protrusions on the mandrel is less than the number of the plurality of score lines formed on the introducer sheath.

26. The method of claim 25, wherein the introducer hub includes a number of notches.

27. The method of claim 26, wherein the number of notches is equal to the number of the plurality of score lines formed on the introducer sheath.

28. The method of claim 27, wherein at least one notch of the number of notches is aligned with one score line of the plurality of score lines formed on the introducer sheath.

29. The method of claim 28, wherein the introducer hub includes at least a first notch and a second notch, the first notch being aligned with a first score line of the plurality of score lines formed on the introducer sheath, and the second notch being aligned with a second score line of the plurality of score lines formed on the introducer sheath.

30. The method of claim 29, wherein the introducer hub and the introducer sheath are both configured to be broken into at least two pieces along the first and second notches and the first and second score lines.

31. The method of claim 24, wherein the number of the plurality of surface protrusions is greater than the number of score lines on the introducer sheath.

32. The method of claim 25, wherein the surface protrusions are aligned along a longitudinal axis.

33. The method of claim 25, wherein the surface protrusions are oriented at an angle to a longitudinal axis.

34. The method of claim 25, wherein the surface protrusions are aligned in a spiral orientation around the mandrel.
